(19) United States
(12) Patent Application Publication
(10) Pub. No.: US 2007/0104702 A1
(43) Pub. Date: May 10, 2007

(54) Method for treating diseases associated with changes of qualitative and/quantitative composition of blood extracellular dna

(76) Inventors: Dmitry Dmitrievich Genkin, Saint-Petersburg (RU); Viktor Veniaminovich Tets, Saint-Petersburg (RU); Georgy Viktorovich Tets, Saint-Petersburg (RU)

Correspondence Address: PATENT, COPYRIGHT & TRADEMARK LAW GROUP, 430 WHITE POND DRIVE, SUITE 200, AKRON, OH, 44320, US

(21) Appl. No.: 10/564,609

(22) PCT Filed: Jul. 1, 2004
(86) PCT No.: PCT/RU04/00260

§ 371 Date: Jan. 12, 2006

(30) Foreign Application Priority Data
Jul. 14, 2003 (RU) PCT/RU03/00304
Mar. 12, 2004 (RU) 2004108057

(51) Int. Cl. A61K 38/46

(52) U.S. Cl. 424/94.6

(57) ABSTRACT

The invention relates to medicine and veterinary science and can be used for treating diseases associated with changes of the qualitative and/quantitative composition of blood extracellular DNA, namely generalised infection diseases provoked by bacteria, diseases provoked by fungi and protozoa, atherosclerosis, pancreatic diabetes, allergic diseases associated with delayed response hypersensitivity and diseases due to somatic cell gene mutations. The inventive method for treating diseases associated with modifications of the qualitative and/or quantitative composition of blood extracellular DNA, namely generalised infection diseases provoked by bacteria, diseases provoked by fungi and protozoa, atherosclerosis, pancreatic diabetes, allergic diseases associated with delayed response hypersensitivity and diseases due to somatic cell gene mutations consists in injecting an agent destroying blood extracellular DNA. DNAse enzyme injected into a systemic blood circulation in doses which modify the electrophoretic profile of the blood extracellular DNA definable by pulse-electrophoresis can be used in the form of an agent destroying said blood extracellular DNA. Said DNAse enzyme can be injected in doses and at regimes ensuring the level of a blood plasma DNA-hydrolytic activity which is measured in the blood plasma and is higher than 150 Kunz units per litre of plasma during a total time higher than 12 hours a day. The inventive method makes it possible to develop a high-efficient and low-toxic method for treating diseases associated with modifications of qualitative and/or quantitative composition of blood extracellular DNA individually or in combination thereof.

### TECHNICAL FIELD

The invention reveals to medicine and veterinary and can be used for treatment of the diseases that are accompanied by quantitative and qualitative changes of blood extracellular DNA in particular: generalized infectious caused by bacteria, diseases caused by fungi and protozoa, atherosclerosis, diabetes, deseases , concerned with delayed-type hypersensitivity reaction and also diseases caused by mutations in somatic cells' genes.

### BACKGROUND ART

According to current knowledge the diseases listed above represent deseases that are extremely different in etiology and pathogenesis. According to this this concepts the treatment of these diseases was carried out by absolutely different methods. Thus the main method of therapy of diseases caused by bacteria, fungi and protozoa are antibiotics and chemotherapy (see Merck Manual of Diagnosis and Therapy; 16th Edition). The main way of atherosclerosis' drug therapy is therapy with statins' group's compounds inhibiting the cholesterol synthesis (see New Concepts and Paradigms in Cardiovascular Medicine: The Noninvasive Management of Coronary Artery Disease, K. Lance Gould, THE AMERICAN JOURNAL OF MEDICINE, Volume 104, Jun. 22, 1998, 2s-17s.)

Therapy of diabetes mellitus consists from three main approaches—insulin therapy, drugs increasing insulin' secretion by pancreas, drugs increasing sensitivity of tissues to the insulin or the same increasing glucose' utilization by tissues (Pharmacological Management of Diabetes: Recent Progress and Future Perspective in Daily Drug Treatment, Gérard Emilien et. al., Pharmacol. Ther. Vol. 81, No. 1, pp. 37-51, 1999).Therapy of type IV hypersensitivity is based on immunosuppressive and immunomodulating therapy (see Therapeutic Immunosupression, ed. A. W. Thomson, Ser. Immunology and Medicine vol. 29, Kluwer Acad. Publishers, Dordrecht, 2001).

The diseases caused by mutations in somatic genes and accompanied by somatic mosaicism development have got no etiological therapy, see Youssoufian H, Pyeritz R E. Mechanisms and Consequences of Somatic Mosaicism in Humans,. Nature Reviews Genetics, 2002; 3:748-758.

The drug resistance is considered as the main problems of the antibiotic therapy of bacterial infection. The circulation of antibiotic-resistant strains and appearance of new ones in the process of the treatment (for example as a result of biofilms' formation in the patient's organism) are the main cause of therapy's inefficiency (The use and resistance to antibiotics in the community. Cizman M, Int J Antimicrob Agents, 2003, April 21: pp. 297-307).

At the present time it is universally recognized that problem of antibiotic resistance has a character of global threat (Mechanisms of antimicrobial resistance: their clinical relevance in the new millennium. Sefton A. M., Drugs, 2002, vol. 62: 557-66) and it asks for development of new original antibiotics and new method with non-antibiotic mechanism of effect on the infectious process. For example vancomycin is used for infectious' treatment caused be Gram positive cocci resistant to penicillin and cephalosporin. Main disadvantages of Vancomycin are increasing of circulating number of Vancomycin-resistant strains; high toxicity; relatively narrow spectrum of activity (The threat of vancomycin resistance. Perl T M, Am J Med 1999 May 106:26S-37S).

The aforesaid data indicate that development new effective, low toxic, method demonstrated broad-spectrum activity against all species bacteria including antibiotic-resistan strains is still an extremely important task. Problems of antibiotic therapy and chemotherapy of infectious caused by fungi and protozoa are close to those of bacterial infection treatment; for example, when an established drug—amphotericin is used (Antifungal drug resistance to azoles and polyenes, Mar Masiá Canuto et. al., The Lancet Infectious Diseases, Volume 2, Issue 9, 1 Sep. 2002, Pages 550-563; A systematic review of the antifungal effectiveness and tolerability of amphotericin B formulations, Jane P. Barrett et. al., Clinical Therapeutics , Volume 25, Issue 5, May 2003, Pages 1295-1320).

Atherosclerosis is a systemic disease that is accompanied by formation of specific atherosclerotic plaques in large and middle sized artery walls. Depends on the localization, stage and size of atherosclerotic plaques the disease has different clinical signs (angina, stroke and so on). The signs especially associated with organ dysfunction caused by systemic atherosclerosis are cured by drug therapy or by surgical operation. There is no cure for Aterosclerosis by drug therapy methods as for any systemic disease. An established method of prevention that delays the disease progression is therapy with inhibitors of 3 3-hydroxy-3-methylglutaryl-CoA (HMG CoA) reductase (Lovastatin, Parvastatine e.t.c.) leading to the inhibition of endogenous cholesterol synthesis and increasing of clearance of low density lipoproteins of blood plasma and it attenuates atherosclerosis development (New Concepts and Paradigms in Cardiovascular Medicine: The Noninvasive Management of Coronary Artery Disease, K. Lance Gould, THE AMERICAN JOURNAL OF MEDICINE, Volume 104, Jun. 22, 1998, 2s-17s). Disadvantages of such treatment are adverse effects (A safety look at currently available statins., Moghadasian M H, Expert Opin Drug Saf 2002 September 1:pp. 269-74) and limited efficacy (Statins: balancing benefits, efficacy and safety., Clearfield M B, Expert Opin Pharmacother, 2002, May 3:pp. 469-77).

The main cause of disablement and death of patients with diabetes mellitus type 1 and 2 are complications associated with microangiopathy and macroangiopathy development. It is considered that effective metabolic control of glucose level (maintenance of glucose level and glycated hemoglobin level within the normal limits) prevents development of complications. The insulin therapy including intensive insulin therapy is the method of choice when it is impossible to reach metabolic control with other drugs (Outpatient insulin therapy in type 1 and type 2 diabetes mellitus: scientific review., DeWitt D E, Hirsch I B, JAMA, 2003, May 289:pp. 2254-64).

But even if high-dose insulin therapy are used the risk of developing the complications including fatal ones is still sufficiently high (Cause-specific mortality in a population with diabetes: South Tees Diabetes Mortality Study., Roper N A, et.al., Diabetes Care, 2002, January 25:pp. 43-8). In accordance with above-mentioned the task of development of new methods of type I and type II diabetes mellitus therapy including the complication prevention methods is still topical and generally recognized.

One of the established clinical methods of treatment of r delayed-type hypersensitivity reactionsis administration of Cyclosporine A peptide (Therapeutic Immunosupression, ed. A. W. Thomson, Ser. Immunology and Medicine vol. 29, Kluwer Acad. Publishers, Dordrecht, 2001). The well-known drawbacks of this method are severe adverse effects namely nephrotoxicity, hypertension and high risk of infections' development (Cyclosporine: mechanisms of action and toxicity., Graham R M, Cleve Clin J Med, 1994, July-August 61:pp. 308-13). Another problem is loss the efficacy during long-term treatment, that reveals itself in the increasing risk for transplant rejection (Renal transplantation, past, present and future., Ponticelli C, et. al., J Nephrol, 1999, July-August 12 Suppl 2:S105-10). Thus for treatment accompanied by qualitative and/or quantitative changes of blood extracellular DNA the wide spectra of different method are used that have got similar disadvantages: toxicity, adverse effects; low efficacy of therapy. At the same time in the real clinical practice these diseases often accompany each other. For example the therapy delayed-type hypersensitivity reactions with immunosuppressive drugs multiplies the risk of infectious diseases (Recent advances in the diagnosis and management of infection in the organ transplant recipient. Tolkoff-Rubin N E, Rubin R H; Semin Nephrol 2000 March 20:148-63.); atherosclerosis is a very common complication of diabetes mellitus (Diabetes and atherosclerosis: epidemiology, pathophysiology, and management. Beckman J A, Creager M A, Libby P; JAMA 2002 May 287:2570-81.) and is often accompanied by systemic infectious process (Infection and atherosclerosis: potential roles of pathogen burden and molecular mimicry., Epstein S E, Zhu J, Burnett M S, Zhou Y F, Vercellotti G, Hajjar D, Arterioscler Thromb Vasc Biol 2000 June 20:1417-20.); a number of diabetes types develops as a result of delayed-type hypersensitivity reaction (Evidence of islet cell autoimmunity in elderly patients with type 2 diabetes., Pietropaolo M, Barinas-Mitchell E, Pietropaolo S L, Kuller L H, Trucco M, Diabetes 2000 January 49:32-8), or during the infectious process (Systemic diseases caused by oral infection. Li X, Kolltveit K M, Tronstad L, Olsen I, Clin Microbiol Rev 2000 October 13:547-58), and leads to the high risk of infections' development (Diabetes and the risk of infection-related mortality in the U.S., Bertoni A G, Saydah S, Brancati F L, Diabetes Care 2001 June 24:6 1044-9).

At the present time there is no single method for the treatment all of diseases listed above. Thus it makes impossible to take any technical solution as prototype.

### DISCLOSURE OF THE INVENTION

The solving of the aim of development of high-performance and low-toxic method of treatment of the diseases that are accompanied by quantitative and/or qualitative change of composition of blood plasma extracellular DNA namely generalized infectious caused by bacteria, diseases caused by fungi and protozoa, atherosclerosis, diabetes, delayed-type hypersensitivity reactions and diseases caused by mutations in genes of somatic cells, presented as a separate disease as their complex, is the basis of this invention.

According to the invention this task is resolved by introducing of blood extracellular DNA destroying agent into a systemic blood circulation for treating diseases associated with changes of qualitative and/quantitative composition of blood extracellular DNA that is observed, namely, generalized infectious caused by bacteria, diseases caused by fungi and protozoa, atherosclerosis, diabetes, delayed-type hypersensitivity reactions and diseases caused by mutations in genes of somatic cells: as the agent restroyng blood extracellular DNA DNase enzyme can be introduced in systemic circulation: enzyme DNAase can be introduced in systemic circulation in doses that provide change of electroforetic profile of blood extracellular DNA that can be detected by puls-gel-electroforesis; DNase enzyme can be administrated at doses and regimens that can provide blood DNA-hydrolytic level measured in blood plasma and exceeded 150 Kuntz units per liter of plasma, and this level can be supported for more than 12 hours during 24 hours in total

Development of diseases listed above is accompanied by quantitative and/or qualitative change of blood extracellular DNA, but in source of available data, there are no knowledge about genetic repertoire of extracellular blood DNA of patients with above-mentioned diseases, about biological role of extracellular blood DNA in these diseases and about potential therapeutic effect of blood extracellular DNA destroying with the purpose of treatment of these diseases; so, taking into account all aforesaid, the invention conformances to requirements of “novelty” criteria (N).

As the applicant established, the extracellular blood DNA of patients with foregoing diseases contains the unique quantitative and qualitative repertoire of genes and regulating genetic elements which great differ from the repertoire of DNA which is described in human genome. In contrast to inracellular DNA the extracellular DNA of these patients contains mainly unique human genes. Extracellular bacterial and fungal DNA was found out in biofilms' matrix and blood plasma of the infected human.

It was established that blood extracellular DNA including extracellular DNA of bacteria, fungi and protozoa promotes development of diseases listed above.

It was established that destruction of blood extracellular plasma DNA leads to therapeutic effect on the diseases listed above.

Aforesaid new characteristics of the claimed invention are based on new ideas about mechanisms of described diseases. In this way the claimed method conformances to requirements of “invention step” criteria (IS).

### BRIEF DESCRIPTION OF THE DRAWINGS

As set for below the invention has been explained by detailed description of embodiments without references to drawings.

### PREFERRED EMBODIMENT

The claimed inventive method is realized by following:
Materials and methods.

The following agents that destroy extracellular blood DNA were used: bovine pancreatic DNase (Sigma and Samson-Med), recombinant human DNase I (Gentech), DNA-hydrolyzing anti-DNA antibodies isolated from the blood of patients with lupus erythematosus according to Shuster A. M. (Shuster A. M. et. al., Science, v.256, 1992, pp. 665-667).

Extracellular DNA from blood plasma was isolated as follows: fresh plasma with (no more than 3-4 hours after sampling) with an added anticoagulant (sodium citrate) was centrifuged on Ficoll-PlaquePlus (Amersham-Pharmacia) during 20 minutes at 1500 g at room temperature. ½ of plasma was detached, not affecting the rest of cells on the Ficoll pillow, and further centrifuged at 10000 g during 30 min for separation from cell fragments and debris. Supernatant was detached, without affecting of the sediment, and was toped up to 1% of sarkosil, 50MM tris-HCl, pH 7,6, 20 MM EDTA, 400 MM NaCl, and than mixed with equal volume of phenol-chloroform (1:1) mixture. The prepared emulsion was incubated during 2 hours at t=65° C., then phenol-chloroform mixture was separated by centrifuging (500 g during 20 minutes, room temperature).

The procedure of deproteinization with phenol-chlorophorm mixture was repeated 3 times, and then the water phase was processed with chloroform and diethyl ether. Separation from organic solvents was made by centrifugation at 5000 g during 15 minutes). Then equal volume of izopropanol was added to resulting aqueous phase and the mixture was incubated overnight at 0° C. After sedimentation the nucleic acids were separated by centrifugation at 10000 g during 30 minutes. The sediment of nucleic acids was dissolved in of 10MM tris-HCl buffer, pH 7, 6 with 5 MM EDTA, and inflicted to the CsCl gradient (1M, 2.5M, 5.7M) in test-tube for rotor SW60Ti. The volume of DNA solution was 2 ml, volume of each step of CsCl was 1 ml. Ultracentrifugation was conducted in L80-80 (Beckman) centrifuge during 3 hours at 250000 g. DNA was collected from the surface of each gradient step into fractions. These fractions were dialyzed during 12 hours (t=4° C.). Presence of DNA in fractions was determined by agar electrophoresis and DNA was visualized by ethidium bromide staining. The amount of DNA was determined with specrophotometer (Beckman DU70) in cuvet (100 mcl) at wavelength of 220-230 nm.

### EXAMPLE 1

Treatment of the experimental sepsis caused by Candida Albicans [symbol] St. Aureus.

Group 1-30 mice were retroorbitally inoculated with 1×10¹⁰ bacteria of pathogenic VT-2003R strain of Staphylococcus aureus. Recombinant dornase-alpha (Genentech) was intraperitoneally administered at dose of 500 mkg/kg in 2, 6, 10 and 14 hours after the inoculation.

Group 2-10 mice mice were retroorbitally inoculated with 1×10¹⁰ bacteria of pathogenic VT-2003R strain of Staphylococcus aureus. Phosphate buffer was intraperitoneally administrated in 2, 6, 10 and 14 hours after the contamination.

After last inoculation of dornase, 24 mice of group 1 were divided to two subgroups (1a and 1b).

Subgroup 1a (8 mice)—2 hours after the last domase administration mice was intravenous injected (at dose 0.1 mkg per animal) with blood extracellular DNA isolated from a number of another mice which were retroorbitally inoculated with 1×10¹⁰ bacteria of pathogenic VT-2003R strain of Staphylococcus aureus 15 hours before the DNA isolation.

Subgroup 1b (8 mice)—2 hours after the last domase administration mice was intravenous injected (at dose 0.1 mkg per animal) with blood extracellular DNA isolated from a number of another mice which were intravenous infecteted with LD50 dose bacteria of Candida Albicans 3 day before the DNA isolation.

The animal's viability was evaluated 32 hours after contamination Results are presented in Table 1

TABLE 1
The mice viability at different time
periods following contamination

| | 0 h. | 2 h. | 4 h. | 6 h. | 8 h. | 12 h. | 24 h. | 28 h. | 32 h. |
|---|---|---|---|---|---|---|---|---|---|
| Group | 100% | 100% | 100% | 90% | 90% | 80% | 50% | 40% | 30% |
| 1 | | | | | | | | | |
| Group | 100% | 100% | 70% | 60% | 50% | 40% | 30% | 20% | 20% |
| 2 | | | | | | | | | |
| 1 a | | | | | | | 20% | 10% | 10% |
| 1 B | | | | | | | 50% | 50% | 40% |

Group 3-10 mice. Clinical isolate of Candida Albicans at LD 50 dose was intravenously administered. Recombinant Dornase alpha (Genentech) was intraperitoneally administered at 1 mg/kg dose twice a day on day 2, day 3 and day 4 after contamination.

Group 4-10 mice. Clinical isolate of Candida Albicans at LD 50 dose was intravenously administered. Amphotericin B was intraperitoneally administered at 20 mg/kg dose twice a day on day 2, day 3 and day 4 after contamination.

Group 5-10 mice. Clinical isolate of Candida Albicans was intravenously administered at LD 50 dose. Phosphate buffer was intraperitoneally administered as negative control twice a day on day 2, day 3 and day 4 contamination.

Viability of mice and their weight was estimated on the 7th day after contamination. Results are presented in the table 2.

TABLE 2
The mice viability at different time
periods following contamination

| | 1 day | 3 day | 5 day | 7 day |
|---|---|---|---|---|
| Group 3 | 100% | 100% | 100% | 100% |
| Group 4 | 100% | 100% | 100% | 100% |
| Group 5 | 100% | 80% | 50% | 50% |

Weight of mice from group 4 on the 7th day of experiment was 20% less than in group 3. This fact indicates that amfotericin B is more toxic than dornase alpha, though their protective efficacies are equal.

Thus blood extracellular DNA of the infected animals possess negative influence on the development of the infectious process and according to the claimed method its destruction is effective in the treatment of bacterial and fungi infections.

### EXAMPLE 2

### Treatment of the Generalized Infection (Sepsis)

38-years-old man has been admitted to the Department of Internal Medicine in grave condition. 12 days before acute respiratory syndrome was diagnosed him. Because of sub febrile temperature, asthenic syndrome and pain in the right chest half, pneumonia was diagnosed 5 days before hospitalization. Cefazoline injection and Roxitomicine per os were prescribed, but no improvement was observed, and two day before admittance to the hospital, fever (39.5-40° C.), sickness, headache were developed. Numerous hemorrhagic rush on the skin, muscle pain, jaundice and diarrhea appeared in the last day before hospitalization. To the time of admittance to the hospital, temperature was 38.3 C, arterial pressure was 100/60; tachycardia of 120 beats per minute, negative meningeal symptoms, cold and cyanotic limbs were found out. Data of laboratory examination: moderate leucocytosis and left shift of hemogram (18% of young forms) was present, appearance of neutrophils with toxic granulation, increasing of conjugated bilirubin, AST and ALT. Numerous small zones of heterogeneity were found at liver' ultrasound examination. The patient was diagnosed as having sepsis. Bacteriological control of blood was done. Gentamicin/Ampicillin/Metronidazole, heparin, vasodilators were per-orally used for treatment. In spite of antimicrobial polychemotherapy, hemabsorption and blood plasma transfusion patient' condition has become worse. Vancomycin' infusions were prescribed as S. Pneumonia was isolated from the patient' blood. During next 48 hours in spite of Vancomycin's infusions condition of patient continued to become worse. Symptoms of multiple organ failure have appeared. Intravenous non-stop infusions of bovine pancreatic DNase at 800 mg/day (1 600 000 Kunitz units) dose were started after parents' approbation. Symptoms of condition' stabilization have appeared twelve hours after initiation of DNase infusion. They were: improvement of peripheral blood circulation and indexes of systemic haemodynamic, appearance of urination. DNase' infusions continued during next 5 days. At that time patient laboratory indexes, haemodynamic, renal function become normal and patient was transferred on self-dependent breathing.

Thus DNase' use according to the claimed method possess therapeutic effect at systemic bacterial infection.

### EXAMPLE 3

### Treatment of Cerebral Malaria

40 mice C57BI have intraperitoneally administered with injection of the erythrocytes, obtained from BALB/c line mice previously infected with P. Bergehi (10⁶ erythrocytes per mice).

Group 1-10 mice were being intramuscularly administered with recombinant dornase-alpha (Genentech) at 500 mkg/kg dose at 24 after contamination and further four times a day for three consecutive days.

Group 2-10 mice were being intramuscularly administered with phosphate buffer.

Group 3-10 mice were being intramuscularly administered with recombinant dornase-alpha (Genentech) at 500 mkg/kg dose at 24 after contamination and further four times a day for three consecutive days. Next day the mice was intravenous injected with (0.1 mkg per mice) blood extracellular DNA isolated from a number of another C57BL mice which were intravenous contaminated with P. Bergehi 5 day before the DNA isolation.

Group 3-10 mice were being intramuscularly administered with recombinant dornase-alpha (Genentech) at 500 mkg/kg dose at 24 after contamination and further four times a day for three consecutive days. Next day the mice was intravenous injected with (0.1 mkg per mice) blood extracellular DNA isolated from a number of another intact C57BL mice.

Viability of mice in experimental and control group was estimated on the 7th day after contamination. Results are presented in the table 3.

TABLE 3
Viability of mice on the 7th day after contamination

| | Group 1 | Group 2 | Group 3 | Group 4 |
|---|---|---|---|---|
| Viability, % | 90 | 10 | 50 | 80 |

Thus extracellular blood plasma DNA of animals contaminated by malaria, negatively influence on the course of infection process and its destruction according to the claimed invention possess therapeutic effect at protozoa infection.

### EXAMPLE 4

### Influence of DNase Therapy on the Viability of Pancreatic Beta-cells and Endothelium of Aorta

Human recombinant DNase I (Gentech) was used. \tilde{β} cells of human embryonic pancreas and endothelial cells of human aorta were used for primary cell culture formation. DNA isolated from plasma of patient with severe form of diabetes mellitus type 2 that was complicated by atherosclerosis (0.0025 mkg on 1 ml of culture media) was added to one of the experimental series in cell culture 24 hours after passage and DNA extracted from the blood of the same patient but treated by DNase (1 mkg/ml; 37 C; 30 minutes) was added to the second series of cell culture. The number of viable cells was counted using the trypan blue uptake technique in a 24 hours.

Results of the experiment are presented in table 4:

TABLE 4
Percentage of viable cells 48 hours after
their cultivation (in percents).

| | | DNA of | DNA of patent |
|---|---|---|---|
| Cells | Control | patient | treated by DNse |
| β cells | 73% | 43% | 61% |
| Endotelium | 62% | 35% | 55% |

Thus extracellular blood plasma DNA of patient with severe form of diabetes mellitus type 2 and atherosclerosis negatively influence both on the normal pancreatic β-cells and on the normal endothelial cells.

Destruction of the patient' blood extracellular DNA prevents development of negative influence according to the claimed method.

### EXAMPLE 5

### Atherosclerosis' Treatment

54-years-old man has been admitted to the hospital in grave condition complaining on intensive pain in abdomen, diarrhea, intensive pain in legs that appear during walking, loss of weight. Diabetes mellitus type 2 was diagnosed 12 years ago and glybencamyde was prescribed. Pain in epigastrium after food intake appeared 15 months ago. Antacids were prescribed but pain continued to increase and steatorrhea appeared in the last 3 months. Because of intensive pain syndrome anorexia has developed in a couple of days before hospitalization. Considerable exhaustion (body weight was 44 kg; body weight loss was 28 kg for the last 5 months) and absence of aorta' pulsation on legs were found out during examination.

No organic changes were observed during gastroduodenoscopy and colonoscopy. Data of ECG was not changed pathologically. Moderate increase of cholesterol level and low-density lipoprotein fraction was observed in blood analysis. Glycated hemoglobin' level was 11%. Partial occlusion of aorta below renal artery (70%), partial occlusion of iliac arteries (90%), total occlusion of upper and lower mesenteric artery were observed on aortography. Conservative therapy was chosen due to absence of possibility to use surgical methods of treatment. Intensive parenteral nutrition was started. Insulin therapy was prescribed. The antiaggregants' therapy have being carried out. By patient's consent daily intravenous infusions of bovine pancreatic DNase at dose 800 mg/day (1 600 000 Kuntz units) divided to 4 two-hour administration were started On the 7th day after the beginning of the therapy patient was allowed to take dietetic food. Pain syndrome disappeared. Patient has received full value enteral nutrition to the 20th day of therapy. General state has improved, body weight has increased. On the 45th day of the therapy angiography as a part preoperative study was done. Decrease of occlusion level of aorta and iliac arteries on 20% and 30% correspondently, and appearance of blood circulation in upper and lower mesenteric artery (occlusion 80%) was observed.

Samples of extracellular DNA of this patient were cloned by the method that allows to construct not amplified plasma library of such DNA with representativeness up to million clones with average size 300-500 base.

Isolated according to above-mentioned method, DNA was deproteinized with the use of proteinase K (Sigma) at 65° C. for tightly-bound proteins elimination. After deproteinization DNA was treated by phenol-chloroform at 65° C. and sedimented by 2,5 volumes of ethanol during night. After it DNA was processed by EcoRI restrictase during 3 hours or by Pfu polymerase (Stratagene) at the presence of 300 mkM of all desoxynucleothydethreephosphates for “sticky” edges elimination. Completed DNA was phosphorylated by polynucleotide kinase T4 (30U, 2 hours). Received samples/preparations were ligated in Bluescript (Stratagene), plasmid digested by EcoRI or PvuII accordingly and dephosphorylated by alkaline phosphatase CIP (Fermentas) during 1 hour. 1 mkg of vector and 0.1-0.5 mkg of serum DNA were usually used for ligation. The ligation was done by Rapid Ligation Kit (Roche) use for 10 hours at 16° C. Volume of ligase mixture was 50 mkl. Ligated library was transformed into DH12S (Life Technologies) cells with electroporator (BioRad) use. For transformation of one library 12-20 electroporation cuvettes were used. Dilutions of the library at concentrations 10⁻⁴, 10⁻⁵ and 10⁻⁶ were plated for control on dishes with 1.5% agar and LB media. In both cases library's representativeness was approximately 2-3×10⁶ clones.

Analysis of randomly chosen clones with length from 300 up to 1000 base pair units from the library that was obtained from the blood extracellular plasma DNA of patient before treatment has indicated that 56 from 75 clones are unique fragments of human DNA. The function or the product of correspondent gene were identified for 11 genes by HumanGeneBank.

| | |
|---|---|
| Gene or corresponding | Reported role in Atherosclerosis and Diabetes |
| protein product | |
| Neutral endopeptidase | At atherosclerosis its activity is increased in |
| | endothelial cells, nonstriated muscle cells, |
| | stromal cells of artery' intima. Decreasing of |
| | its activity can decrease lipids accumulation |
| | in vessels wall. |
| Muskelin 1 | Is mediator of cell response on |
| | thrombospondin 1. thrombospondin 1 - |
| | mediated processes are pathophysiological |
| | components of atherosclerotic affection of |
| | artery wall. |
| Nf-kappaB | At hyperglycemia and atherosclerosis its |
| | activity is increased in cells of artery wall. |
| Transient receptor | |
| potential cation | |
| channel | |
| Phospholipase C, | Induces expression of receptors of low |
| epsilon | density lipoproteins. |
| CRTL1: cartilage | |
| linking protein 1 | |
| 17 kD fetal brain | |
| protein | |
| Nicotinamide | |
| nucleotide | |
| transhydrogenase | |
| BAI3: brain-specific | |
| angiogenesis inhibitor | |
| GAD2: glutamate | One of the main autoantigens at diabetes type |
| decarboxylase 2 | 1. |
| E-selectin | High level of expression is a risk factor of |
| | angiopathy' development at diabetes type 2. |

Analysis of 50 clones randomly chosen from the library obtained from the extracellular blood plasma DNA of patient on the 21^{st} day after the beginning of treatment has shown that more than 90% of revealed clone consequences are short fragments of repeating human DNA, in the main alpha-satellite DNA. Changes in electroforetic profile of blood extracellular DNA that is evaluated by pulse gel electrophoresis were registered at the same time.

Thus DNase use according to the claimed method possess therapeutic effect at atherosclerosis.

### EXAMPLE 6

### Diabetes Mellitus Treatment

Poor metabolic control of diabetes mellitus, that is revealed by high level of glycated hemoglobin in blood and low sensitivity to insulin makes it necessary to use high doses of insulin and are main predisposing factors to the complications' development.

46-years-old patient suffers from diabetes mellitus type 2 for 3 years. Because of the unsuccessfully results of oral antidiabetic compounds that were not able to decrease blood glucose level, the human recombinant short-acting insulin was prescribed at 0.3 U/kg (21U/day). The level of glycated hemoglobin in blood was still to be high (more than 10%), symptoms of diabetic angiopathy and polyneuropathy and decrease in sharpness of vision have appeared. Daily insulin demand has increased up to 1.2 U/kg (84 U/day). Intramuscular injections of bovine pancreatic DNase at 200 mg/day (2 times a day) for 4 months were prescribed to the patient. To the end of treatment course the patient was better , level of glycated hemoglobin in blood was diminished and daily dose of insulin was also diminished in two times. The results are presented in table 5.

TABLE 5
The effect of DNase treatment on patient' metabolic indexes.

| | | Three | |
|---|---|---|---|
| | | months after | Four months |
| | | the | after the |
| | Before | beginning of | beginning of |
| | treatment | treatment | treatment |
| Glycated hemoglobin | 13.2 | 10.1 | 7.2 |
| (%) | | | |
| Necessity for insulin | 1.2 | 0.9 | 0.6 |
| U/kg body weight | | | |
| Amount of extracellular | 100 | 85 | 70 |
| blood plasma DNA | | | |
| (%). | | | |

Amount of blood extracellular DNA was estimated by densitometry of electrophoretic band. DNA amount before treatment was taken as 100%.

Thus DNase use at diabetes possess therapeutic effect according to the claimed method.

### EXAMPLE 7

### Inhibition of Activation for Lymphocytes

20 mice C57BI were transcutaneously immunized by Mycobacterium Smegmatis suspension (100 mkg of antigen in 50 mkl of aluminium alum) in the foot pincushion. Four weeks later mice were killed and splenocytes were isolated. Splenocytes of the sensibilized and intact mice were cultivated Petry dishes in suspension culture (2.5×10⁶ cells/ml) in PRPMI 1640 media with 2mM Glutamine, antibiotics and 10% Fetal calf serum in the presence of Mycobacterium Smegmatis (5 mkg/ml) antigen, for 24 hours at 5% CO2 at 37° C. For determination of splenocytes' activation level in the presence of antigen [3H]-thymidine up to 0.1 mCi/ml concentration was added 6 hours before end of cultivating. After the cultivation cells were washed up, dissolved in formamide and radioactivity was measured.

Series 1 (5 dishes) Splenocytes of sensibilized mice.

Series 2 (5 dishes) Splenocytes of not sensibilized mice. Recombinant dornase-alpha (Genentech) was added at 1 mkg/ml concentration to the media.

Series 3 (5 dishes) Splenocytes of intact mice.

Series 4 (5 dishes) Splenocytes of intact mice. Blood extracellular DNA isolated from sensibilized mice 2 hour after repeated subcutaneous injection of Mycobacterium Smegmatis antigen at 200 mkg dose was added to the medium. DNA was added at 0.05 mkg/ml concentration.

Series 5 (5 dishes) Splenocytes of intact mice. Blood extracellular DNA isolated from intact mice 2 hour after repeated subcutaneous injection of Mycobacterium Smegmatis antigen at 200 mkg dose was added to the medium. DNA was added at 0.05 mkg/ml concentration

Series 6 (5 dishes). Splenocytes of intact mice that were cultivated without antigen adding.

Uptake of [3H] thymidine by splenocytes 24 hours after cultivatiion with antigen.

The results of the experiment are presented in table 6.

TABLE 6
Inhibition of lymphocytes' activation.

| N of series | Lymphocytes' number (CPM) |
|---|---|
| 1 | 115000 |
| 2 | 75000 |
| 3 | 35000 |
| 4 | 95000 |
| 5 | 40000 |
| 6 | 15000 |

Thus blood extracellular plasma DNA increase lymphocytes' specific activation under antigen stimulation and DNase use leads to the inhibition of antigenic stimulation according to claimed method.

### EXAMPLE 8

### Treatment of Delayed-type Hypersensitivity

23-years-old man has been admitted to the hospital in grave condition. Chronicle mieloleucosis was diagnosed 4 years ago in 1999. Therapy with hydroxyurea and alpha-interferon were previously held. Bone marrow transplantation was done because of disease's acute progression. Bone marrow was transplanted from HLA compatible but ABO incompatible donor, and total body irradiation with the following cyclophosphan administration was performed Patient administrated methotrexat for graft-versus-host reaction' prevention. On the ninth day graft-versus-host reaction with generalized rush and diarrhea has developed. Patient has received pulse therapy of methylprednisolone and antilymphocitaric globulin for 9 days. Patient's condition has improved. To the 30th day function of bone marrow has restored and patient was discharged from the hospital. One week later patient has been repeatedly admitted to the hospital with leucopenia (leucocytes were 0.9*10⁹), ulceration on the oral cavity mucosa and fever. Hypoplasia and eosinophilia were found at aspiration biopsy. Azathioprine and leucomax was prescribed but 6 weeks later leucopenia (leucocytes 0.7*10⁹) has developed. Azathioprine was withhold and puls-therapy with methylprednisolone and leucomax was done. At the same time fever, ulcerations on the oral cavity mucosa was still not to be eliminated.

Soon after the end of therapy, episodes of intravascular hemolysis with reduction of leucocyte' and thrombocytes' number have happened. Intravenous infusions of bovine pancreatic desoxiribonuclease at 400 mg/day (800 000 Kuntz units) dose 6 times a day for one hour during 2 weeks were prescribed to the patient. Blood plasma level of DNA hydrolysis activity was more than 180 Kuntz units per liter of plasma during not less than 12 hours. Starting from the 5th day of therapy patient's condition has improved. Number of leucocytes to the 7th day has increased up to 1.7*10⁹, and to the 15th day averaged 2.4*10⁹. To that time hemolysis symptoms have disappeared, temperature decreased and ulcers' sanitation in the mouth was observed. Oral cavity ulcers was sanified and Number of erythrocites normalized. Patient was discharged from the hospital in satisfactory condition. One month later normal blood formula was observed during control visit to hospital.

Thus DNase use according to claimed method possess therapeutic effect at delayed-type hypersensitivity reaction.

### EXAMPLE 9

### Inhibition of Mutant Gene Spreading

Some human diseases evolve from somatic mosaicism' development. Somatic mosaicism is an expansion of mutant gene in population of somatic cells (Youssoufian H, Pyeritz R E. Mechanisms and Consequences of Somatic Mosaicism in Humans. Nature Reviews Genetics 2002;3:748-758.)

Frequency of HPRT gene's mutations in blood T-lymphocytes was studied as a model of somatic mosaicism. Human HPRT gene (chromosome Xq26) encodes enzyme that is constitutionally expressing but not essential and is involved in metabolism of purine basis. Cloning was done according to Bigbee W. method (Bigbee W. Et al., Mutation Res., 1998, v. 397, pp. 119-136). For cloning were used peripheral blood lymphocytes of 8 cancer patients that previously administered by 3 week course of immunostimulant Neovir after surgical elimination of tumor. 4 patients from this group (8 patients) have additionally intravenously administrated therapy by human recombinant DNase I (200 mkg/kg dose 4 times a day during three weeks). The rate of HPRT-deficient clones in the blood of patients that received DNase I therapy was 3 times less than the same in blood of patients that received only immunopotentiating therapy. Addition of the blood extracellular DNA from patients ,who were not subjected to DNase administration, to the culture medium during cloning of T-lymphocytes from patients subjected to DNase administration, increases frequency of HPRT-deficient clones appearance.

Thus destruction of patient' blood extracellular plasma DNA by DNase enzyme according to the claimed method prevents development of somatic mosaicism.

### EXAMPLE 10

### Elimination of Pathogenic Properties of Extracellular DNA by Different Methods

Mice C57B1 were inoculated with highly metastatic LLC tumor strain. On the 9th day after inoculation animals were killed under anaesthetic and total blood plasma was taken. The total fraction of blood extracellular DNA was stored at −20° C. in phosphate buffer.

Six groups of mice that participated in the experiment were inoculated with metastatic LLC strain.

Group 1-6 mice inoculated with low metastatic LLC strain.

Group 2-6 mice inoculated with low metastatic LLC strain that additionally intravenously administered twice (on the 7th and 8th day after the inoculation) with DNA fraction of mice previously inoculated with highly metastatic strain (0.05 mkg of DNA was dissolved in 500 mkl of fresh heparinized blood).

Group 3-6 mice inoculated with low-metastatic LLC strain that additionally intravenously administered twice (on the 7th and 8th day after the inoculation) with DNA fraction of mice previously inoculated with high-metastatic strain (0.05 mkg of DNA was dissolved in 500 mkl of fresh heparinized blood). Before administration the DNA sample was photochemically disinfected (1 mkM of methylene blue was added with following irradiation by red light during 10 minutes (approx. 60 000 Lux).

Group 4-6 mice inoculated with low-metastatic LLC strain that additionally intravenously administered twice (on the 7th and 8th day after the inoculation) with DNA fraction of mice previously inoculated with high-metastatic strain (0.05 mkg of DNA was dissolved in 500 mkl of fresh heparinized blood). The DNA sample was mixed with 10 mkg ant-DNA hydrolizing antibodies before administration.

Group 5-6 mice inoculated with low-metastatic LLC strain that additionally intravenously administered twice (on the 7th and 8th day after the inoculation) with DNA fraction of mice previously inoculated with high-metastatic strain (0.05 mkg of DNA was dissolved in 500 mkl of fresh heparinized blood). Before the administration 1 mkg of the fragment A of the plant toxin Ricin was added to the sample and the mixture was incubated during 1 hour at =37° C. Ricin is the representative of RIP-toxins family (proteins inactivating ribosomes) which widely used for immunotoxins' development . In addition to its capability to inactivate ribosomes these proteins also can deadenilate and hydrolyze DNA. To realize of the toxic effect the unit A of the type II RIP toxin should be delivered into cell by unit B. In absence of subunit B chain A is not toxic, however polyadeninglicosidase activity of chain A can be used for destruction of DNA circulating in plasma.

Group 7-6 mice inoculated with low-metastatic LLC strain that additionally intravenously administered twice (on the 7th and 8th day after the inoculation) with DNA fraction of mice previously inoculated with low-metastatic strain (0.05 mkg of DNA was dissolved in 500 mkl fresh heparinized blood).

Number of lungs' metastaic lesions was estimated on the 15th day after inoculation.

The results of the experiments are presented in the table 7.

TABLE 7
Number of metastatic lesions (N) in lungs
on the 15th day after replantation

| Group | |
|---|---|
| number | N |
| 1 | 12.0 |
| 2 | 22.5 |
| 3 | 14.1 |
| 4 | 15.5 |
| 5 | 15.1 |
| 6 | 13.3 |

Thus different methods of extracellular DNA' destruction inhibit pathogenic properties.

### INDUSTRIAL APPLICABILITY

For the realization the methods there were used well-known materials and equipment manufactured in plant conditions and according to aforesaid the invention conformances to requirements of “industrial applicability” criteria (IA).

1. A method for treating diseases associated with changes of qualitative and/quantitative composition of blood extracellular DNA in particular: generalized infectious caused by bacteria, diseases caused by fungi and protozoa, atherosclerosis, diabetes, delayed-type hypersensitivity reaction and diseases caused by mutations in somatic cells' genes characterized by introducing of blood extracellular DNA destroying agent into a systemic blood circulation.

2. A method according to claim 1 wherein DNAase enzyme is introduced into a systemic blood circulation.

3. A method according to claim 1 which characterized by introducing of DNAase enzyme in doses sufficient to provide blood extracellular DNA electrophoretic profile change, which can be revealed by pulse-gelelectrophoresis.

4. A method according to claim 1 characterized by introducing of DNAase enzyme in doses and regimens which provide blood plasma DNA-hydrolytic activity, measured in blood plasma, to exceed 150 Kunitz units per liter of plasma during more then 12 hours in total within 24 hours.
